(19) United States
(12) Patent Application Publication
(10) Pub. No.: US 2010/0049322 A1
(43) Pub. Date: Feb. 25, 2010

(54) OSTEOCHONDRAL REPAIR IMPLANTS AND METHODS

(75) Inventor: William F. MCKAY, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, TN (US)

(21) Appl. No.: 12/193,794

(22) Filed: Aug. 19, 2008

(51) Int. Cl. A61F 2/28; A61F 2/02; A61B 17/08; A61B 19/00

(52) U.S. Cl. 623/16.11; 623/23.72; 606/151; 128/898

(57) ABSTRACT

An osteochondral repair implant is provided, comprising a tissue scaffold configured to allow growth of at least bone and/or cartilage and to fit within at least a portion of an osteochondral hole, the tissue scaffold comprising one or more recesses and/or projections; and a biodegradable carrier containing a growth factor, the biodegradable carrier being placed in and/or around the recesses and/or projections of the tissue scaffold and reduce compression of the biodegradable carrier by the tissue scaffold. In some embodiments, methods for repairing an osteochondral defect are provided that utilize an osteochondral plug and a biodegradable carrier containing a growth factor.

### BACKGROUND

Joint surfaces are covered by articular cartilage that provides a resilient, durable surface with low friction. Cartilage is an avascular tissue that has a small number of chondrocytes encapsulated within an extensive extracellular matrix. The cartilage acts to distribute mechanical forces and to protect subchondral bone. The knee is a particular instance of a cartilage surfaced (the condyle) bone area. The knee comprises three bones—the femur, tibia, and patella that are held in place by various ligaments. Corresponding chondral areas of the femur and the tibia form a hinge joint and the patella protects the joint. Portions of the chondral areas as well as the underside of the patella are covered with an articular cartilage that allows the femur and the tibia to smoothly glide against each other without causing damage.

Damage to the articular cartilage, subchondral bone or both can result from traumatic injury or a disease state and can be extremely painful to the patient. For example, articular cartilage in the knee can tear due to traumatic injury as with athletes and degenerative processes as with older patients. The knee cartilage does not heal well due to lack of nerves, blood vessels and a lymphatic system. Hyaline cartilage in particular has a limited capacity for repair and lesions in this material without intervention can form repair tissue lacking the biomechanical properties of normal cartilage.

A number of procedures are used to treat damaged articular cartilage. Currently, the most widely used procedure involves lavage, arthroscopic debridement and repair stimulation. Typically, repair stimulation is conducted by drilling, abrasion arthroplasty or microfracture. The goal of this procedure is to penetrate into subchondral bone to induce bleeding and fibrin clot formation. This promotes initial repair. However, the resulting formed tissue is often fibrous in nature and lacks the durability of normal cartilage.

Osteochondral grafting has been used to repair chondral damage and to replace damaged articular cartilage and subchondral bone. Typically, in this procedure, cartilage and bone tissue of a defect site are removed by routing to create a bore of a cylindrical geometry. Then a tissue scaffold such as a cylindrical cartilage and subchondral bone plug graft is harvested in a matching geometry. The harvest is typically from another body region of less strain (e.g., hip, skull, ribs, etc.), called autograft, or from bone taken from other people that is frozen and stored in tissue banks, called allograft or from animals of a different species called xenograft. The harvested plug graft is then implanted into the bore of the routed defect site. Healing of the graft bone to host bone results in fixation of the plug graft to surrounding host region.

Some grafting procedures utilize a variety of natural and synthetic tissue scaffolds with or instead of bone (e.g., collagen, silicone, acrylics, hydroxyapatite, calcium sulfate, ceramics, etc.), which are press-fit into the osteochondral hole at a patient's defect area.

Sometimes growth factors (e.g., bone morphogenic protein) may be introduced on a carrier into the osteochondral hole and then when the tissue scaffold is press-fit into the osteochondral hole, the surgeon will use such force that will cause the tissue scaffold to over compress the carrier and cause an excess amount of the growth factor to leak from the carrier, which may reduces a stable microenvironment for new bone and/or cartilage growth. Thus, there is a need to develop new osteochondral repair implants and methods.

### SUMMARY

Compositions and methods are provided that improve osteochondral repair. Through the use of these new osteochondral repair implants, the growth of bone, cartilage and/or related tissue may be facilitated particularly when repairing osteochondral defects. In some embodiments, the osteochondral repair implants reduce unwanted compression of the biodegradable carrier when the tissue scaffold is inserted into the osteochondral defect. In some embodiments, “compression resistant” osteochondral plugs are provided that reduce growth factor leakage from a biodegradable carrier on insertion of the plug into the osteochondral defect.

In some embodiments, the present application can reduce or prevent compression of the tissue scaffold from occurring that causes the growth factor to be forced into surrounding subchondral bone which can lead to local transient bone resorption. It can also prevent buffer from the bone growth factor to leak from the carrier, which causes a higher concentration of the growth factor (e.g., rhBMP-2) to remain on the carrier. This high concentration growth factor may lead to local transient bone resorption. In some embodiments, the present application can reduce or prevent some compression of the tissue scaffold from occurring that may cause the growth factor to contact the tissue scaffold causing a low dose of growth factor (e.g., rhBMP-2) to be absorbed into the upper portion of the scaffold to promote cartilage formation at the site the tissue scaffold is implanted.

In some embodiments, an osteochondral repair implant is provided, comprising a tissue scaffold configured to allow growth of at least bone and/or cartilage and to fit within at least a portion of an osteochondral hole, the tissue scaffold comprising one or more mating recesses and/or projections; and a biodegradable carrier containing a growth factor, the biodegradable carrier having one or more opposing mating recesses and/or projections configured to receive the one or more mating recesses and/or projections of the tissue scaffold and reduce compression of the biodegradable carrier by the tissue scaffold.

In some embodiments, an osteochondral repair implant is provided, comprising a tissue scaffold configured to allow growth of at least bone and/or cartilage and to fit within at least a portion of an osteochondral hole, the tissue scaffold comprising one or more mating recesses and/or projections; and a biodegradable carrier containing a growth factor, the biodegradable carrier being placed in and/or around the one or more opposing mating recesses and/or projections configured to receive the one or more mating recesses and/or projections of the tissue scaffold and reduce compression of the biodegradable carrier by the tissue scaffold.

In some embodiments, an osteochondral repair implant is provided, comprising a osteochondral plug configured to allow growth of at least bone and/or cartilage and to fit within at least a portion of an osteochondral hole, the osteochondral plug comprising one or more recesses and/or projections; and a biodegradable carrier containing a growth factor, the biodegradable carrier being placed in and/or around the recesses and/or projections of the osteochondral plug to reduce compression of the biodegradable carrier by the osteochondral plug and leakage of the growth factor from the biodegradable carrier.

In some embodiments, a method is provided for repairing an osteochondral defect in a patient in need of such treatment, the method comprising: forming a hole in the bone and/or cartilage in an osteochondral area in need of repair, the hole having an upper portion configured to receive a tissue scaffold and a lower portion configured to receive a biodegradable carrier containing a growth factor; inserting into the hole the biodegradable carrier so as to fill part or all of the lower portion of the hole with the biodegradable carrier and inserting into the hole a tissue scaffold so as to fill part or all of the upper portion of the hole with the biodegradable carrier to stack the tissue scaffold on the biodegradable carrier without compressing the biodegradable carrier and causing substantial leakage of the growth factor therefrom.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE FIGURES

In part, other aspects, features, benefits and advantages of the embodiments will be apparent with regard to the following description, appended claims and accompanying drawings where:

FIG. 1A is an illustration of a top view and a side sectional view of an osteochondral repair implant as it is being implanted into an osteochondral hole.

FIG. 1B is an illustration of a side sectional view of an osteochondral repair implant as it is implanted into an osteochondral hole. The tissue scaffold (e.g., shown as a plug) compresses against the biodegradable carrier upon implantation causing the growth factor or an aqueous buffer to be squeezed out or leak out from the carrier.

FIG. 2A is an illustration of a top view and a side sectional view of an osteochondral hole that is drilled around the osteochondral defect. The osteochondral hole has geometric sizes in an upper portion that fits at least a portion of the tissue scaffold (e.g., shown as a plug) snuggly within the upper portion of the osteochondral hole. The lower portion has an area that fits the biodegradable carrier.

FIG. 2B is an illustration of a side sectional view of the osteochondral repair implant that is implanted into an osteochondral hole. The osteochondral hole has geometric sizes in an upper portion that fits snuggly at least a portion of the tissue scaffold (e.g., shown as a plug) within the upper portion of the osteochondral hole. The lower portion has an area that fits the biodegradable carrier. In this way, the user upon forcing the tissue scaffold into the osteochondral hole cannot overly compress the biodegradable carrier causing unwanted growth factor or buffer leakage into the surrounding tissue.

FIG. 3 is an illustration of a side sectional view of the osteochondral repair implant that is implanted into an osteochondral hole. In this view, the tissue scaffold (e.g., shown as a tapered plug) fits snuggly within the osteochondral hole. Since the tissue scaffold is tapered, the user cannot force the tissue scaffold into the osteochondral hole and thus cannot overly compress the biodegradable carrier causing unwanted growth factor or buffer leakage into the surrounding tissue.

FIG. 4A is an illustration of a top view of an osteochondral hole around the osteochondral defect. FIG. 4B illustrates an embodiment of the tissue scaffold (e.g., shown as a plug) that has one mating recess and two mating projections configured to receive the biodegradable carrier that contains a growth factor. FIG. 4C is a side sectional view of the osteochondral repair implant that is implanted into an osteochondral hole of a cartilage surface. In this view, the tissue scaffold (e.g., shown as a plug) having one mating recess and two mating projections receives the biodegradable carrier. In this way the surface area of the collagen carrier that is exposed to compressive forces upon insertion is less. Thus, it is much more difficult to compress the biodegradable carrier to cause unwanted growth factor or buffer leakage into the surrounding tissue.

FIG. 5A is an illustration of a top view of an osteochondral hole around the osteochondral defect. FIG. 5B illustrates an embodiment of the tissue scaffold (e.g., shown as a plug) that has two mating recesses and one mating projection configured to receive the biodegradable carrier that contains a growth factor. FIG. 5C is a side sectional view of the osteochondral repair implant that is implanted into an osteochondral hole of a cartilage surface. In this view, the tissue scaffold (e.g., shown as a plug) having two mating recesses and one mating projection receives the two projections of the biodegradable carrier. In this way, the surface area of the collagen carrier that is exposed to compressive forces upon insertion is less. Thus, it is much more difficult to compress the biodegradable carrier causing unwanted growth factor or buffer leakage into the surrounding tissue.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present application. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical are as precise as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

Additionally, unless defined otherwise or apparent from context, all technical and scientific terms used herein have the same meanings as commonly understood by one of ordinary skill in the art to which this invention belongs.

Unless explicitly stated or apparent from context, the following terms are phrases have the definitions provided below:

### DEFINITIONS

It is noted that, as used in this specification and the appended claims, the singular forms “a,” “an,” and “the,” include plural referents unless expressly and unequivocally limited to one referent. Thus, for example, reference to “a tissue scaffold” includes one, two, three or more tissue scaffolds.

The term “biodegradable” includes that all or parts of the carrier and/or scaffold will degrade over time by the action of enzymes, by hydrolytic action and/or by other similar mechanisms in the human body. In various embodiments, “biodegradable” includes that the carrier and/or scaffold can break down or degrade within the body to non-toxic components after or while a therapeutic agent has been or is being released. By “bioerodible” it is meant that the carrier and/or scaffold will erode or degrade over time due, at least in part, to contact with substances found in the surrounding tissue, fluids or by cellular action. By “bioabsorbable” or “bioresorbable” it is meant that the carrier and/or scaffold will be broken down and absorbed within the human body, for example, by a cell or tissue. “Biocompatible” means that the carrier and/or scaffold will not cause substantial tissue irritation or necrosis at the target tissue site.

The term “mammal” refers to organisms from the taxonomy class “mammalian,” including but not limited to humans, other primates such as chimpanzees, apes, orangutans and monkeys, rats, mice, cats, dogs, cows, horses, etc.

“A “therapeutically effective amount” or “effective amount” is such that when administered, the drug (e.g., growth factor) results in alteration of the biological activity, such as, for example, promotion of bone, cartilage and/or other tissue (e.g., vascular tissue) growth, inhibition of inflammation, reduction or alleviation of pain, improvement in the condition through inhibition of an immunologic response, etc. The dosage administered to a patient can be as single or multiple doses depending upon a variety of factors, including the drug's administered pharmacokinetic properties, the route of administration, patient conditions and characteristics (sex, age, body weight, health, size, etc.), extent of symptoms, concurrent treatments, frequency of treatment and the effect desired. In some embodiments the formulation is designed for immediate release. In other embodiments the formulation is designed for sustained release. In other embodiments, the formulation comprises one or more immediate release surfaces and one or more sustained release surfaces.

The phrase “immediate release” is used herein to refer to one or more therapeutic agent(s) that is introduced into the body and that is allowed to dissolve in or become absorbed at the location to which it is administered, with no intention of delaying or prolonging the dissolution or absorption of the drug.

The phrases “sustained release” and “sustain release” (also referred to as extended release or controlled release) are used herein to refer to one or more therapeutic agent(s) that is introduced into the body of a human or other mammal and continuously or continually releases a stream of one or more therapeutic agents over a predetermined time period and at a therapeutic level sufficient to achieve a desired therapeutic effect throughout the predetermined time period.

The terms “treating” and “treatment” when used in connection with a disease or condition refer to executing a protocol that may include osteochondral repair procedure, administering one or more drugs to a patient (human, other normal or otherwise or other mammal), in an effort to alleviate signs or symptoms of the disease or condition or immunological response. Alleviation can occur prior to signs or symptoms of the disease or condition appearing, as well as after their appearance. Thus, treating or treatment includes preventing or prevention of disease or undesirable condition. In addition, treating, treatment, preventing or prevention do not require complete alleviation of signs or symptoms, does not require a cure, and specifically includes protocols that have only a marginal effect on the patient. In some embodiments, the osteochondral repair implant can be used to treat subchondral, osteochondral, hyaline cartilage and/or condyle defects.

The term “subchondral” includes an area underlying joint cartilage. The term “subchondral bone” includes a very dense, but thin layer of bone just below a zone of cartilage and above the cancellous or trabecular bone that forms the bulk of the bone structure of the limb. “Osteochondral” includes a combined area of cartilage and bone where a lesion or lesions can occur. “Osteochondral defect” includes a lesion which is a composite lesion of cartilage and subchondral bone. “Hyaline cartilage” includes cartilage containing groups of isogenous chondrocytes located within lacunae cavities which are scattered throughout an extracellular collagen matrix. A “condyle” includes a rounded articular surface of the extremity of a bone.

The phrase “osteogenic composition” refers to a composition that comprises a substance that promotes bone growth.

Reference will now be made in detail to certain embodiments of the invention. While the invention will be described in conjunction with the illustrated embodiments, it will be understood that they are not intended to limit the invention to those embodiments. On the contrary, the invention is intended to cover all alternatives, modifications, and equivalents that may be included within the invention as defined by the appended claims.

Compositions and methods are provided that improve osteochondral repair. Through the use of these new osteochondral repair implants, the growth of bone, cartilage and/or related tissue may be facilitated particularly when repairing osteochondral defects. In some embodiments, the osteochondral repair implants reduce unwanted compression of the biodegradable carrier when the tissue scaffold is inserted into the osteochondral defect. In some embodiments, “compression resistant” osteochondral plugs are provided that reduce growth factor leakage from a biodegradable carrier on insertion of the plug into the osteochondral defect.

In some embodiments, an osteochondral repair implant is provided, comprising a tissue scaffold configured to allow growth of at least bone and/or cartilage and to fit within at least a portion of an osteochondral hole, the tissue scaffold comprising one or more mating recesses and/or projections; and a biodegradable carrier containing a growth factor, the biodegradable carrier having one or more opposing mating recesses and/or projections configured to receive the one or more mating recesses and/or projections of the tissue scaffold and reduce compression of the biodegradable carrier by the tissue scaffold.

The headings below are not meant to limit the disclosure in any way; embodiments under any one heading may be used in conjunction with embodiments under any other heading.

### Tissue Scaffold

The tissue scaffolds provides a matrix for the cells to guide the process of tissue formation in vivo in three dimensions. The morphology of the scaffold guides cell migration and cells are able to migrate into or over the scaffold, respectively. The cells then are able to proliferate and synthesize new tissue and form bone and/or cartilage. In some embodiments, one or more tissue scaffolds are stacked on one or more biodegradable carriers.

In some embodiments, the tissue scaffold comprises a plurality of pores. In some embodiments, at least 10% of the pores are between about 10 micrometers and about 500 micrometers at their widest points. In some embodiments, at least 20% of the pores are between about 50 micrometers and about 150 micrometers at their widest points. In some embodiments, at least 30% of the pores are between about 30 micrometers and about 70 micrometers at their widest points. In some embodiments, at least 50% of the pores are between about 10 micrometers and about 500 micrometers at their widest points. In some embodiments, at least 90% of the pores are between about 50 micrometers and about 150 micrometers at their widest points. In some embodiments, at least 95% of the pores are between about 100 micrometers and about 250 micrometers at their widest points. In some embodiments, 100% of the pores are between about 10 micrometers and about 300 micrometers at their widest points.

In some embodiments, the tissue scaffold has a porosity of at least about 30%, at least about 50%, at least about 60%, at least about 70%, at least about 90%. The pore may support ingrowth of cells, formation or remodeling of bone, cartilage and/or vascular tissue.

The tissue scaffold may comprise natural and/or synthetic material. For example, the tissue scaffold may comprise poly (alpha-hydroxy acids), poly (lactide-co-glycolide) (PLGA), polylactide (PLA), polyglycolide (PG), polyethylene glycol (PEG) conjugates of poly (alpha-hydroxy acids), polyorthoesters (POE), polyaspirins, polyphosphagenes, collagen, hydrolyzed collagen, gelatin, hydrolyzed gelatin, fractions of hydrolyzed gelatin, elastin, starch, pre-gelatinized starch, hyaluronic acid, chitosan, alginate, albumin, fibrin, vitamin E analogs, such as alpha tocopheryl acetate, d-alpha tocopheryl succinate, D,L-lactide, or L-lactide, ,-caprolactone, dextrans, vinylpyrrolidone, polyvinyl alcohol (PVA), PVA-g-PLGA, PEGT-PBT copolymer (polyactive), methacrylates, poly (N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate), polydioxanone, methylmethacrylate (MMA), MMA and N-vinylpyyrolidone, polyamide, oxycellulose, copolymer of glycolic acid and trimethylene carbonate, polyesteramides, polyetheretherketone, polymethylmethacrylate, or combinations thereof.

In some embodiments, the tissue scaffold may comprise a resorbable ceramic (e.g., hydroxyapatite, tricalcium phosphate, bioglasses, calcium sulfate, etc.) tyrosine-derived polycarbonate poly(DTE-co-DT carbonate), in which the pendant group via the tyrosine—an amino acid—is either an ethyl ester (DTE) or free carboxylate (DT) or combinations thereof.

In some embodiments, the tissue scaffold comprises collagen. Exemplary collagens include human or non-human (bovine, ovine, and/or porcine), as well as recombinant collagen or combinations thereof. Examples of suitable collagen include, but are not limited to, human collagen type I, human collagen type II, human collagen type III, human collagen type IV, human collagen type V, human collagen type VI, human collagen type VII, human collagen type VIII, human collagen type IX, human collagen type X, human collagen type XI, human collagen type XII, human collagen type XIII, human collagen type XIV, human collagen type XV, human collagen type XVI, human collagen type XVII, human collagen type XVIII, human collagen type XIX, human collagen type XXI, human collagen type XXII, human collagen type XXIII, human collagen type XXIV, human collagen type XXV, human collagen type XXVI, human collagen type XXVII, and human collagen type XXVIII, or combinations thereof. Collagen further may comprise hetero- and homo-trimers of any of the above-recited collagen types. In some embodiments, the collagen comprises hetero- or homo-trimers of human collagen type I, human collagen type II, human collagen type III, or combinations thereof.

In some embodiments, the embodiments the tissue scaffold may comprise particles of bone-derived materials. The bone-derived material may include one or more of non-demineralized bone particles, demineralized bone particles, lightly demineralized bone particles, and/or deorganified bone particles.

In some embodiments, the tissue scaffold may be seeded with harvested bone cells and/or bone tissue, such as for example, cortical bone, autogenous bone, allogenic bones and/or xenogenic bone. In some embodiments, the tissue scaffold may be seeded with harvested cartilage cells and/or cartilage tissue (e.g., autogenous, allogenic, and/or xenogenic cartilage tissue). For example, before insertion into the target tissue site, the tissue scaffold can be wetted with the graft bone tissue/cells, usually with bone tissue/cells aspirated from the patient, at a ratio of about 3:1, 2:1, 1:1, 1:3 or 1:2 by volume. The bone tissue/cells are permitted to soak into the scaffolding provided, and the scaffolding may be kneaded by hand, thereby obtaining a pliable consistency that may subsequently be packed into the osteochondral defect. In some embodiments, the tissue scaffold provides a malleable, non-water soluble carrier that permits accurate placement and retention at the implantation site.

The scaffold may contain an inorganic material, such as an inorganic ceramic and/or bone substitute material. Exemplary inorganic materials or bone substitute materials include but are not limited to aragonite, dahlite, calcite, amorphous calcium carbonate, vaterite, weddellite, whewellite, struvite, urate, ferrihydrate, francolite, monohydrocalcite, magnetite, goethite, dentin, calcium carbonate, calcium sulfate, calcium phosphosilicate, sodium phosphate, calcium aluminate, calcium phosphate, hydroxyapatite, alpha-tricalcium phosphate, dicalcium phosphate, β-tricalcium phosphate, tetracalcium phosphate, amorphous calcium phosphate, octacalcium phosphate, BIOGLASS™, fluoroapatite, chlorapatite, magnesium-substituted tricalcium phosphate, carbonate hydroxyapatite, substituted forms of hydroxyapatite (e.g., hydroxyapatite derived from bone may be substituted with other ions such as fluoride, chloride, magnesium sodium, potassium, etc.), or combinations or derivatives thereof.

In some embodiments, tissue will infiltrate the scaffold to a degree of about at least 50 percent within about 1 month to about 6 months after implantation of the scaffold. In some embodiments, about 75 percent of the scaffold will be infiltrated by tissue within about 2-3 months after implantation of the scaffold. In some embodiments, the scaffold will be substantially, e.g., about 90 percent or more, submerged in or enveloped by tissue within about 6 months after implantation of the scaffold. In some embodiments, the scaffold will be completely submerged in or enveloped by tissue within about 9-12 months after implantation.

In some embodiments, the tissue scaffold has a thickness of from 0.25 mm to 5 mm, or from about 0.4 mm to about 2 mm, or 0.4 mm to about 1 mm. Clearly, different osteochondral defects may require different tissue scaffold thickness.

In some embodiments, the tissue scaffold has a density of between about 1.6 g/cm³, and about 0.05 g/cm³. In some embodiments, the tissue scaffold has a density of between about 1.1 g/cm³, and about 0.07 g/cm³. For example, the density may be less than about 1 g/cm³, less than about 0.7 g/cm³, less than about 0.6 g/cm³, less than about 0.5 g/cm³, less than about 0.4 g/cm³, less than about 0.3 g/cm³, less than about 0.2 g/cm³, or less than about 0.1 g/cm³.

The shape of the tissue scaffold may be tailored to the site at which it is to be situated. For example, it may be in the shape of a morsel, a plug, a pin, a peg, a cylinder, a block, a wedge, a sheet, etc.

In some embodiments, the diameter or diagonal of the tissue scaffold can range from 1 mm to 30 mm. In some embodiments, the diameter or diagonal of the tissue scaffold can range from 1 mm to 10 mm, or 4 mm to 8 mm which is small enough to fit through an endoscopic cannula, but large enough to minimize the number of plugs needed to fill a large osteochondral hole.

In some embodiments, the tissue scaffold has an upper surface that is contoured to match the bone and/or cartilage surface of the excised osteochondral tissue and can pack well into the osteochondral hole and reduce scaffold rotation.

In some embodiments, the tissue scaffold is configured to fit snugly within the osteochondral hole. In some embodiments, the tissue scaffold is configured to fit within an upper portion of the osteochondral hole so that the bottom portion of the osteochondral hole has sufficient space for placement of the biodegradable carrier. In this way, it is difficult for the surgeon to over compress the biodegradable carrier and cause the growth factor to leak out.

In some embodiments, the tissue scaffold comprises on its side surfaces or bottom surfaces one or more mating recesses and/or projections that are configured to receive the one or more opposing mating recesses and/or projections of the biodegradable carrier. In this way, the surface area of the biodegradable carrier cannot be substantially compressed by the tissue scaffold.

It will be understood by those of ordinary skill in the art that some compression may occur to release the growth factor (e.g., less than 0.75 M Pa, or 0.5 M Pa, or 0.25 M Pa of pressure), which may release less than 10%, 9%, 8%, 7%, 6%, 5%, 4%, 3%, 2%, 1%, or 0.5% w/w or w/v of the growth factor.

In some embodiments, the tissue scaffold can resist compression forces in the range of from about 1 MPa to about 100 MPa.

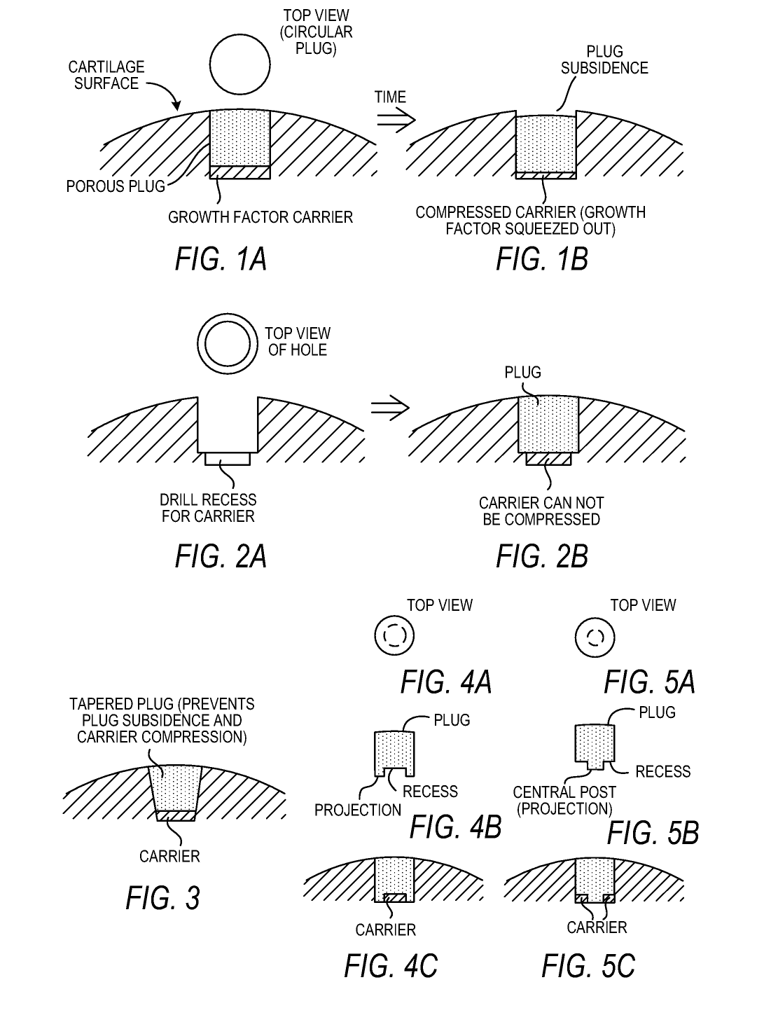

In some embodiments, if a plug or tissue scaffold gets over compressed, the pore's top surface falls below the surface of the cartilage and the surface of the plug is no longer flush with the cartilage surface (shown in FIG. 1B).

In some embodiments, the tissue scaffold may be made by injection molding, compression molding, blow molding, thermoforming, die pressing, slip casting, electrochemical machining, laser cutting, water-jet machining, electrophoretic deposition, powder injection molding, sand casting, shell mold casting, lost tissue scaffold casting, plaster-mold casting, ceramic-mold casting, investment casting, vacuum casting, permanent-mold casting, slush casting, pressure casting, die casting, centrifugal casting, squeeze casting, rolling, forging, swaging, extrusion, shearing, spinning, powder metallurgy compaction or combinations thereof.

In some embodiments, a therapeutic agent (not including a growth factor) may be disposed on or in the tissue scaffold by hand, electrospraying, ionization spraying or impregnating, vibratory dispersion (including sonication), nozzle spraying, compressed-air-assisted spraying, brushing and/or pouring. For example, a growth factor such as rhBMP-2 may be disposed on or in the tissue scaffold.

In some embodiments, the tissue scaffold may comprise sterile and/or preservative free material.

### Plugs

In some embodiments, the tissue scaffold comprises a shaped plug graft (e.g., pin, a peg, a cylinder, trapezoid, a block, a wedge, etc.). In some embodiments, at least one cross sectional profile of the plug graft configuration, tapers from top to bottom surface (FIG. 3).

In some embodiments, an osteochondral plug graft is provided in the form of a biphasic construct that has an upper surfaced cartilage area and a subchondral bone lower portion. The biphasic construct may comprise an articular cartilage top surface with a lower subchondral bone portion having two parallel sides and a bottom surface. The bottom bone surface can be parallel to the articular cartilage top surface. The non parallel subchondral bone sides are tapered from the articular cartilage top surface to permit tight packing of an array of the plug grafts within an osteochondral hole.

In some embodiments, the plug graft can be harvested from a recipient or from another suitable human or animal donor and from any appropriate structure including hyaline cartilage and underlying subchondral bone. Suitable harvest locations include joints of mammals, including humans, such as articulating surfaces of knee, hip and shoulder joints. For example, an osteochondral plug can be harvested at the femoral condyle or the articulating surface of a knee joint.

In some embodiments, the plug graft can be harvested in any shape and manipulated to provide the desired shape and include optionally the desired recesses and/or projections. For example, a cylindrical plug can be reamed from a host osteochondral site and pared into a desired trapezoid shape appropriate for implant into the osteochondral hole. Or, a plug in a trapezoid shape can be harvested with a punch much like the formation of a square or cube with a punch.

In some embodiments, a shaped allograft osteochondral plug is provided that can be either fresh (containing live cells) or processed and frozen or otherwise preserved to remove cells and other potentially antigenic substances while leaving behind a scaffold for patient tissue ingrowth. A variety of such processing techniques is known and can be used in accordance with the present application. For example, harvested osteochondral plugs can be soaked in an agent that facilitates removal of cell and proteoglycan components. One such solution includes an aqueous preparation of hyaluronidase (type IV, 3 mg/ml), and trypsin (0.25% in monodibasic buffer 3 ml). The harvested osteochondral plugs can be soaked in this solution for several hours, for example 10 to 24 hours, desirably at an elevated temperature such as 37° C. Optionally, a mixing method such as sonication can be used during the soak. Additional processing steps can include decalcification, washing with water, and immersion in organic solvent solutions such as chloroform/methanol to remove cellular debris and sterilize. After such immersion the grafts can be rinsed thoroughly with water and then frozen and optionally lyophilized. These and other conventional tissue preservation techniques can be applied to the osteochondral grafts in accordance with the present application.

Osteochondral tissue scaffolds (e.g., graft plugs) can be used in the repair of articular cartilage in a patient including for example, cartilage occurring in weight bearing joints such as the knee. The articular cartilage in need of repair can present a full thickness defect, including damage to both the cartilage and the underlying subchondral bone. Such defects can occur from trauma or an advanced stage of disease, including an arthritic disease.

Typically, an articular cartilage site in need of treatment will be surgically prepared for receipt of the osteochondral plug graft. This preparation can include excision of cartilage and subchondral bone at the site to create a hole or void in which the plug graft will be received. Tissue removal can be conducted in any suitable manner including for instance drilling and/or punching, typically in a direction substantially perpendicular to the articular cartilage layer at the site, to create a recipient hole or void having a depth approximating that of the graft to be implanted. In some embodiments, the hole will have an upper portion that will have a surface large enough to receive the tissue scaffold. However, the lower portion of the hole will have a surface small enough to receive the biodegradable carrier but little or no tissue scaffold (e.g., FIG. 2A). In some embodiments, the lower hole diameter (shown as a drill recess for carrier in FIG. 2A) can be about 0.5 to about 2 mm smaller than the upper hole diameter. In some embodiments, the upper hole diameter is between about 6 to about 12 mm. By drilling the hole in this configuration, this can prevent the plug from “bottoming out” on insertion. In this way, the surgeon cannot over compress the tissue scaffold on the biodegradable carrier and release the growth factor. In some embodiments, the depth of the lower hole can be 1 mm to 3 cm and the upper hole 3-15 mm.

In some embodiments, the depth of the tissue scaffold can be for example, 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm.

In some embodiments, the opening for receiving the graft will be created using a drill or punch having a circular cross-section. In some embodiments, multiple, overlapping passes with the drill or punch can be made, in order to create an opening having a cross-section defined by multiple, intersecting circular arcs. A chisel can then be used to shape the recipient hole to receive the osteochondral implant.

In some embodiments, the tissue scaffold and/or biodegradable carrier can include cells (e.g., bone, chondrogenic cells and/or tissue) seeded or attached to it.

In some embodiments, to treat damaged articular cartilage, a cartilage transplant procedure may be utilized, such as, for example, a Mosaicplasty or osteoarticular transfer system (OATS) technique. Typically, this technique involves using a series of dowel cutting instruments to harvest a plug of articular cartilage and subchondral bone from a donor site, which can then be implanted into a hole or void made into the defect site. By repeating this process, transferring a series of plugs, and by placing them in close proximity to one another, in mosaic-like fashion, a new grafted hyaline cartilage surface can be established. The result is a hyaline-like surface interposed with a fibrocartilage healing response between each graft.

In some embodiments, a small amount of biologic glue can be applied into the osteochondral hole and the plug inserted into the glue-lined hole. Suitable organic glues include TISSEEL® or TISSUCOL® (fibrin based adhesive; Immuno AG, Austria), Adhesive Protein (Sigma Chemical, USA), Dow Coming Medical Adhesive B (Dow Corning, USA), fibrinogen thrombin, elastin, collagen, alginate, demineralized bone matrix, casein, albumin, keratin or the like. A composite fibrin glue can be mixed with milled cartilage from for example, a bovine fibrinogen (e.g., SIGMA F-8630), thrombin (e.g., SIGMA T-4648) and aprotinin (e.g., SIGMA A6012. Also, human derived fibrinogen, thrombin and aprotinin can be used.

In some embodiments, a method is provided for repairing an osteochondral defect in a patient in need of such treatment, the method comprising: forming a hole in the bone and/or cartilage in an osteochondral area in need of repair, the hole having an upper portion configured to receive a tissue scaffold and a lower portion configured to receive a biodegradable carrier containing a growth factor; inserting into the hole the biodegradable carrier so as to fill part or all of the lower portion of the hole with the biodegradable carrier and inserting into the hole a tissue scaffold so as to fill part or all of the upper portion of the hole with the biodegradable carrier to stack the tissue scaffold on the biodegradable carrier without compressing the biodegradable carrier and causing substantial leakage of the growth factor therefrom.

In some embodiments, a method is provided for repairing an osteochondral defect, where the upper portion of the osteochondral hole is wider than the lower portion of the hole so that the tissue scaffold cannot substantially move toward the lower portion of the hole upon pressure to compress the biodegradable carrier.

### Biodegradable Carrier

The biodegradable carrier is a matrix to hold the growth factor and allows it to be released to the surrounding tissue site and/or tissue scaffold to exert its effect. In some embodiments, it is the first component that is inserted into the osteochondral hole.

Like the tissue scaffold, the biodegradable carrier provides a matrix for the cells to guide the process of tissue formation in vivo in three dimensions. The morphology of the biodegradable carrier guides cell migration and cells are able to migrate into or over the carrier, respectively. The cells then are able to proliferate and synthesize new tissue and form bone and/or cartilage.

In some embodiments, the biodegradable carrier comprises a plurality of pores. In some embodiments, at least 10% of the pores are between about 10 micrometers and about 500 micrometers at their widest points. In some embodiments, at least 20% of the pores are between about 50 micrometers and about 150 micrometers at their widest points. In some embodiments, at least 30% of the pores are between about 30 micrometers and about 70 micrometers at their widest points. In some embodiments, at least 50% of the pores are between about 10 micrometers and about 500 micrometers at their widest points. In some embodiments, at least 90% of the pores are between about 50 micrometers and about 150 micrometers at their widest points. In some embodiments, at least 95% of the pores are between about 100 micrometers and about 250 micrometers at their widest points. In some embodiments, 100% of the pores are between about 10 micrometers and about 300 micrometers at their widest points.

In some embodiments, the biodegradable carrier has a porosity of at least about 30%, at least about 50%, at least about 60%, at least about 70%, at least about 90%. The pores may support ingrowth of cells, formation or remodeling of bone, cartilage and/or vascular tissue.

The biodegradable carrier may comprise natural and/or synthetic material. For example, the biodegradable carrier may comprise poly (alpha-hydroxy acids), poly (lactide-co-glycolide) (PLGA), polylactide (PLA), polyglycolide (PG), polyethylene glycol (PEG) conjugates of poly (alpha-hydroxy acids), polyorthoesters (POE), polyaspirins, polyphosphagenes, collagen, hydrolyzed collagen, gelatin, hydrolyzed gelatin, fractions of hydrolyzed gelatin, elastin, starch, pre-gelatinized starch, hyaluronic acid, chitosan, alginate, albumin, fibrin, vitamin E analogs, such as alpha tocopheryl acetate, d-alpha tocopheryl succinate, D,L-lactide, or L-lactide, ,-caprolactone, dextrans, vinylpyrrolidone, polyvinyl alcohol (PVA), PVA-g-PLGA, PEGT-PBT copolymer (polyactive), methacrylates, poly (N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate), polydioxanone, methylmethacrylate (MMA), MMA and N-vinylpyyrolidone, polyamide, oxycellulose, copolymer of glycolic acid and trimethylene carbonate, polyesteramides, polyetheretherketone, polymethylmethacrylate, or combinations thereof.

In some embodiments, the biodegradable carrier may comprise a resorbable ceramic (e.g., hydroxyapatite, tricalcium phosphate, bioglasses, calcium sulfate, etc.) tyrosine-derived polycarbonate poly(DTE-co-DT carbonate), in which the pendant group via the tyrosine—an amino acid—is either an ethyl ester (DTE) or free carboxylate (DT) or combinations thereof.

In some embodiments, the biodegradable carrier comprises collagen. Exemplary collagens include human or non-human (bovine, ovine, and/or porcine), as well as recombinant collagen or combinations thereof. Examples of suitable collagen include, but are not limited to, human collagen type I, human collagen type II, human collagen type III, human collagen type IV, human collagen type V, human collagen type VI, human collagen type VII, human collagen type VIII, human collagen type IX, human collagen type X, human collagen type XI, human collagen type XII, human collagen type XIII, human collagen type XIV, human collagen type XV, human collagen type XVI, human collagen type XVII, human collagen type XVIII, human collagen type XIX, human collagen type XXI, human collagen type XXII, human collagen type XXIII, human collagen type XXIV, human collagen type XXV, human collagen type XXVI, human collagen type XXVII, and human collagen type XXVIII, or combinations thereof. Collagen further may comprise hetero- and homo-trimers of any of the above-recited collagen types. In some embodiments, the collagen comprises hetero- or homo-trimers of human collagen type I, human collagen type II, human collagen type III, or combinations thereof.

In some embodiments, the biodegradable carrier may comprise particles of bone-derived materials. The bone-derived material may include one or more of non-demineralized bone particles, demineralized bone particles, lightly demineralized bone particles, and/or deorganified bone particles.

In some embodiments, like the tissue scaffold, the biodegradable carrier may be seeded with harvested bone cells and/or bone tissue, such as for example, cortical bone, autogenous bone, allogenic bones and/or xenogenic bone. In some embodiments, the biodegradable carrier may be seeded with harvested cartilage cells and/or cartilage tissue (e.g., autogenous, allogenic, and/or xenogenic cartilage tissue). For example, before insertion into the target tissue site, the biodegradable carrier can be wetted with the graft bone tissue/cells, usually with bone tissue/cells aspirated from the patient, at a ratio of about 3:1, 2:1, 1:1, 1:3 or 1:2 by volume. The bone tissue/cells are permitted to soak into the biodegradable carrier provided, and the carrier may be kneaded by hand, thereby obtaining a pliable consistency that may subsequently be gently packed into the lower part of the osteochondral defect. In some embodiments, the biodegradable carrier provides a malleable, non-water soluble carrier that permits accurate placement and retention at the implantation site.

In some embodiments, the biodegradable carrier may contain an inorganic material, such as an inorganic ceramic and/or bone substitute material. Exemplary inorganic materials or bone substitute materials include but are not limited to aragonite, dahlite, calcite, amorphous calcium carbonate, vaterite, weddellite, whewellite, struvite, urate, ferrihydrate, francolite, monohydrocalcite, magnetite, goethite, dentin, calcium carbonate, calcium sulfate, calcium phosphosilicate, sodium phosphate, calcium aluminate, calcium phosphate, hydroxyapatite, alpha-tricalcium phosphate, dicalcium phosphate, P-tricalcium phosphate, tetracalcium phosphate, amorphous calcium phosphate, octacalcium phosphate, BIOGLASS™, fluoroapatite, chlorapatite, magnesium-substituted tricalcium phosphate, carbonate hydroxyapatite, substituted forms of hydroxyapatite (e.g., hydroxyapatite derived from bone may be substituted with other ions such as fluoride, chloride, magnesium sodium, potassium, etc.), or combinations or derivatives thereof.

In some embodiments, tissue will infiltrate the carrier to a degree of about at least 50 percent within about 1 month to about 6 months after implantation of the carrier. In some embodiments, about 75 percent of the carrier will be infiltrated by tissue within about 2-3 months after implantation of the carrier. In some embodiments, the carrier will be substantially, e.g., about 90 percent or more, submerged in or enveloped by tissue within about 6 months after implantation of the carrier. In some embodiments, the carrier will be completely submerged in or enveloped by tissue within about 9-12 months after implantation.

In some embodiments, the biodegradable carrier has a thickness of from 0.25 mm to 5 mm, or from about 0.4 mm to about 2 mm, or 0.4 mm to about 1 mm. Clearly, different osteochondral defects may require different biodegradable carrier thickness.

In some embodiments, the biodegradable carrier has a density of between about 1.6 g/cm³, and about 0.05 g/cm³. In some embodiments, the biodegradable carrier has a density of between about 1.1 g/cm³, and about 0.07 g/cm³. For example, the density may be less than about 1 g/cm³, less than about 0.7 g/cm³, less than about 0.6 g/cm³, less than about 0.5 g/cm³, less than about 0.4 g/cm³, less than about 0.3 g/cm³, less than about 0.2 g/cm³, or less than about 0.1 g/cm³.

The shape of the biodegradable carrier may be tailored to the site at which it is to be situated. For example, it may be in the shape of a morsel, a block, a wedge, a sheet, etc. In some embodiments, the biodegradable carrier can have mating recesses and or projections on its top or side surfaces to receive the opposing recesses and/or projections from the tissue scaffold.

In some embodiments, the diameter or diagonal of the biodegradable carrier can range from 1 mm to 50 mm. In some embodiments, the diameter or diagonal of the biodegradable carrier can range from 1 mm to 30 mm, or 5 mm to 10 mm which is small enough to fit through an endoscopic cannula, but large enough to minimize the number of plugs needed to fill a large osteochondral hole.

Exemplary carriers for use in the present application include large and soft collagen sponges available from Medtronic Sofamor Danek (Memphis, Tenn.) as Mastergraft®. In some embodiments, the carrier may comprise sterile and/or preservative free material.

In some embodiments, the biodegradable carrier may be made by injection molding, compression molding, blow molding, thermoforming, die pressing, slip casting, electrochemical machining, laser cutting, water-jet machining, electrophoretic deposition, powder injection molding, sand casting, shell mold casting, lost tissue scaffold casting, plaster-mold casting, ceramic-mold casting, investment casting, vacuum casting, permanent-mold casting, slush casting, pressure casting, die casting, centrifugal casting, squeeze casting, rolling, forging, swaging, extrusion, shearing, spinning, powder metallurgy compaction or combinations thereof.

### Growth Factors

In some embodiments, a growth factor and/or therapeutic agent may be disposed on or in the carrier by hand, electrospraying, ionization spraying or impregnating, vibratory dispersion (including sonication), nozzle spraying, compressed-air-assisted spraying, brushing and/or pouring. For example, a growth factor such as rhBMP-2 may be disposed on or in the biodegradable carrier by the surgeon before the biodegradable carrier is administered or it may be available from the manufacturer beforehand.

The biodegradable carrier may comprise at least one growth factor. These growth factors include osteoinductive agents (e.g., agents that cause new bone growth in an area where there was none) and/or osteoconductive agents (e.g., agents that cause in growth of cells into and/or through the tissue scaffold). Osteoinductive agents can be polypeptides or polynucleotides compositions. Polynucleotide compositions of the osteoinductive agents include, but are not limited to, isolated Bone Morphogenetic Protein (BMP), Vascular Endothelial Growth Factor (VEGF), Connective Tissue Growth Factor (CTGF), Osteoprotegerin, Growth Differentiation Factors (GDFs), Cartilage Derived Morphogenic Proteins (CDMPs), Lim Mineralization Proteins (LMPs), Platelet derived growth factor, (PDGF or rhPDGF), Insulin-like growth factor (IGF) or Transforming Growth Factor beta (TGF-beta) polynucleotides. Polynucleotide compositions of the osteoinductive agents include, but are not limited to, gene therapy vectors harboring polynucleotides encoding the osteoinductive polypeptide of interest. Gene therapy methods often utilize a polynucleotide, which codes for the osteoinductive polypeptide operatively linked or associated to a promoter or any other genetic elements necessary for the expression of the osteoinductive polypeptide by the target tissue. Such gene therapy and delivery techniques are known in the art, (See, for example, International Publication No. WO90/11092, the disclosure of which is herein incorporated by reference in its entirety). Suitable gene therapy vectors include, but are not limited to, gene therapy vectors that do not integrate into the host genome. Alternatively, suitable gene therapy vectors include, but are not limited to, gene therapy vectors that integrate into the host genome.

In some embodiments, the polynucleotide is delivered in plasmid formulations. Plasmid DNA or RNA formulations refer to polynucleotide sequences encoding osteoinductive polypeptides that are free from any delivery vehicle that acts to assist, promote or facilitate entry into the cell, including viral sequences, viral particles, liposome formulations, lipofectin, precipitating agents or the like. Optionally, gene therapy compositions can be delivered in liposome formulations and lipofectin formulations, which can be prepared by methods well known to those skilled in the art. General methods are described, for example, in U.S. Pat. Nos. 5,593,972, 5,589,466, and 5,580,859, the disclosures of which are herein incorporated by reference in their entireties.

Gene therapy vectors further comprise suitable adenoviral vectors including, but not limited to for example, those described in U.S. Pat. No. 5,652,224, which is herein incorporated by reference.

Polypeptide compositions of the isolated osteoinductive agents include, but are not limited to, isolated Bone Morphogenetic Protein (BMP), Vascular Endothelial Growth Factor (VEGF), Connective Tissue Growth Factor (CTGF), Osteoprotegerin, Growth Differentiation Factors (GDFs), Cartilage Derived Morphogenic Proteins (CDMPs), Lim Mineralization Proteins (LMPs), Platelet derived growth factor, (PDGF or rhPDGF), Insulin-like growth factor (IGF) or Transforming Growth Factor beta (TGF-beta707) polypeptides. Polypeptide compositions of the osteoinductive agents include, but are not limited to, full length proteins, fragments or variants thereof.

Variants of the isolated osteoinductive agents include, but are not limited to, polypeptide variants that are designed to increase the duration of activity of the osteoinductive agent in vivo. Preferred embodiments of variant osteoinductive agents include, but are not limited to, full length proteins or fragments thereof that are conjugated to polyethylene glycol (PEG) moieties to increase their half-life in vivo (also known as pegylation). Methods of pegylating polypeptides are well known in the art (See, e.g., U.S. Pat. No. 6,552,170 and European Pat. No. 0,401,384 as examples of methods of generating pegylated polypeptides). In some embodiments, the isolated osteoinductive agent(s) are provided as fusion proteins. In one embodiment, the osteoinductive agent(s) are available as fusion proteins with the Fc portion of human IgG. In another embodiment, the osteoinductive agent(s) are available as hetero- or homodimers or multimers. Examples of some fusion proteins include, but are not limited to, ligand fusions between mature osteoinductive polypeptides and the Fc portion of human Immunoglobulin G (IgG). Methods of making fusion proteins and constructs encoding the same are well known in the art.

Isolated osteoinductive agents that are included within carrier are typically sterile. In a non-limiting method, sterility is readily accomplished for example by filtration through sterile filtration membranes (e.g., 0.2 micron membranes or filters). In one embodiment, the isolated osteoinductive agents include one or more members of the family of Bone Morphogenetic Proteins (“BMPs”). BMPs are a class of proteins thought to have osteoinductive or growth-promoting activities on endogenous bone tissue, or function as pro-collagen precursors. Known members of the BMP family include, but are not limited to, BMP-1, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, BMP-18 as well as polynucleotides or polypeptides thereof, as well as mature polypeptides or polynucleotides encoding the same.

BMPs utilized as osteoinductive agents comprise one or more of BMP-1; BMP-2; BMP-3; BMP-4; BMP-5; BMP-6; BMP-7; BMP-8; BMP-9; BMP-10; BMP-11; BMP-12; BMP-13; BMP-15; BMP-16; BMP-17; or BMP-18; as well as any combination of one or more of these BMPs, including full length BMPs or fragments thereof, or combinations thereof, either as polypeptides or polynucleotides encoding the polypeptide fragments of all of the recited BMPs. The isolated BMP osteoinductive agents may be administered as polynucleotides, polypeptides, full length protein or combinations thereof.

In another embodiment, isolated osteoinductive agents include osteoclastogenesis inhibitors to inhibit bone resorption of the bone tissue surrounding the site of implantation by osteoclasts. Osteoclast and osteoclastogenesis inhibitors include, but are not limited to, osteoprotegerin polynucleotides or polypeptides, as well as mature osteoprotegerin proteins, polypeptides or polynucleotides encoding the same. Osteoprotegerin is a member of the TNF-receptor superfamily and is an osteoblast-secreted decoy receptor that functions as a negative regulator of bone resorption. This protein specifically binds to its ligand, osteoprotegerin ligand (TNFSF11/OPGL), both of which are key extracellular regulators of osteoclast development.

Osteoclastogenesis inhibitors further include, but are not limited to, chemical compounds such as bisphosphonate, 5-lipoxygenase inhibitors such as those described in U.S. Pat. Nos. 5,534,524 and 6,455,541 (the contents of which are herein incorporated by reference in their entireties), heterocyclic compounds such as those described in U.S. Pat. No. 5,658,935 (herein incorporated by reference in its entirety), 2,4-dioxoimidazolidine and imidazolidine derivative compounds such as those described in U.S. Pat. Nos. 5,397,796 and 5,554,594 (the contents of which are herein incorporated by reference in their entireties), sulfonamide derivatives such as those described in U.S. Pat. No. 6,313,119 (herein incorporated by reference in its entirety), or acylguanidine compounds such as those described in U.S. Pat. No. 6,492,356 (herein incorporated by reference in its entirety).

In another embodiment, isolated osteoinductive agents include one or more members of the family of Connective Tissue Growth Factors (“CTGFs”). CTGFs are a class of proteins thought to have growth-promoting activities on connective tissues. Known members of the CTGF family include, but are not limited to, CTGF-1, CTGF-2, CTGF-4 polynucleotides or polypeptides thereof, as well as mature proteins, polypeptides or polynucleotides encoding the same.

In another embodiment, isolated osteoinductive agents include one or more members of the family of Vascular Endothelial Growth Factors (“VEGFs”). VEGFs are a class of proteins thought to have growth-promoting activities on vascular tissues. Known members of the VEGF family include, but are not limited to, VEGF-A, VEGF-B, VEGF-C, VEGF-D, VEGF-E or polynucleotides or polypeptides thereof, as well as mature VEGF-A, proteins, polypeptides or polynucleotides encoding the same.

In another embodiment, isolated osteoinductive agents include one or more members of the family of Transforming Growth Factor-beta genes (“TGFbetas”). TGF-betas are a class of proteins thought to have growth-promoting activities on a range of tissues, including connective tissues. Known members of the TGF-beta family include, but are not limited to, TGF-beta-1, TGF-beta-2, TGF-beta-3, polynucleotides or polypeptides thereof, as well as mature protein, polypeptides or polynucleotides encoding the same.

In another embodiment, isolated osteoinductive agents include one or more Growth Differentiation Factors (“GDFs”). Known GDFs include, but are not limited to, GDF-1, GDF-2, GDF-3, GDF-7, GDF-10, GDF-11, and GDF-15. For example, GDFs useful as isolated osteoinductive agents include, but are not limited to, the following GDFs: GDF-1 polynucleotides or polypeptides corresponding to GenBank Accession Numbers M62302, AAA58501, and AAB94786, as well as mature GDF-1 polypeptides or polynucleotides encoding the same. GDF-2 polynucleotides or polypeptides corresponding to GenBank Accession Numbers BC069643, BC074921, Q9UK05, AAH69643, or AAH74921, as well as mature GDF-2 polypeptides or polynucleotides encoding the same. GDF-3 polynucleotides or polypeptides corresponding to GenBank Accession Numbers AF263538, BC030959, AAF91389, AAQ89234, or Q9NR23, as well as mature GDF-3 polypeptides or polynucleotides encoding the same. GDF-7 polynucleotides or polypeptides corresponding to GenBank Accession Numbers AB158468, AF522369, AAP97720, or Q7Z4P5, as well as mature GDF-7 polypeptides or polynucleotides encoding the same. GDF-10 polynucleotides or polypeptides corresponding to GenBank Accession Numbers BC028237 or AAH28237, as well as mature GDF-10 polypeptides or polynucleotides encoding the same.

GDF-11 polynucleotides or polypeptides corresponding to GenBank Accession Numbers AF100907, NP_{—}005802 or 095390, as well as mature GDF-11 polypeptides or polynucleotides encoding the same. GDF-15 polynucleotides or polypeptides corresponding to GenBank Accession Numbers BC008962, BC000529, AAH00529, or NP_{—}004855, as well as mature GDF-15 polypeptides or polynucleotides encoding the same.

In another embodiment, isolated osteoinductive agents include Cartilage Derived Morphogenic Protein (CDMP) and Lim Mineralization Protein (LMP) polynucleotides or polypeptides. Known CDMPs and LMPs include, but are not limited to, CDMP-1, CDMP-2, LMP-1, LMP-2, or LMP-3.

CDMPs and LMPs useful as isolated osteoinductive agents include, but are not limited to, the following CDMPs and LMPs: CDMP-1 polynucleotides and polypeptides corresponding to GenBank Accession Numbers NM_{—}000557, U13660, NP_{—}000548 or P43026, as well as mature CDMP-1 polypeptides or polynucleotides encoding the same. CDMP-2 polypeptides corresponding to GenBank Accession Numbers or P55106, as well as mature CDMP-2 polypeptides. LMP-1 polynucleotides or polypeptides corresponding to GenBank Accession Numbers AF345904 or AAK30567, as well as mature LMP-1 polypeptides or polynucleotides encoding the same. LMP-2 polynucleotides or polypeptides corresponding to GenBank Accession Numbers AF345905 or AAK30568, as well as mature LMP-2 polypeptides or polynucleotides encoding the same. LMP-3 polynucleotides or polypeptides corresponding to GenBank Accession Numbers AF345906 or AAK30569, as well as mature LMP-3 polypeptides or polynucleotides encoding the same.

In another embodiment, isolated osteoinductive agents include one or more members of any one of the families of Bone Morphogenetic Proteins (BMPs), Connective Tissue Growth Factors (CTGFs), Vascular Endothelial Growth Factors (VEGFs), Osteoprotegerin or any of the other osteoclastogenesis inhibitors, Growth Differentiation Factors (GDFs), Cartilage Derived Morphogenic Proteins (CDMPs), Lim Mineralization Proteins (LMPs), or Transforming Growth Factor-betas (TGF-betas), as well as mixtures or combinations thereof.

In another embodiment, the one or more isolated osteoinductive agents useful in the bioactive formulation are selected from the group consisting of BMP-1, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, BMP-18, or any combination thereof; CTGF-1, CTGF-2, CGTF-3, CTGF-4, or any combination thereof; VEGF-A, VEGF-B, VEGF-C, VEGF-D, VEGF-E, or any combination thereof; GDF-1, GDF-2, GDF-3, GDF-7, GDF-10, GDF-11, GDF-15, or any combination thereof; CDMP-1, CDMP-2, LMP-1, LMP-2, LMP-3, and or combination thereof; Osteoprotegerin; TGF-beta-1, TGF-beta-2, TGF-beta-3, or any combination thereof; or any combination of one or more members of these groups.

The concentrations of growth factor can be varied based on the desired length or degree of osteogenic effects desired. Similarly, one of skill in the art will understand that the duration of sustained release of the growth factor can be modified by the manipulation of the compositions comprising the sustained release formulation, such as for example, modifying the percent of polymers found within a sustained release formulation, microencapsulation of the formulation within polymers, including polymers having varying degradation times and characteristics, and layering the formulation in varying thicknesses in one or more degradable polymers. These sustained release formulations can therefore be designed to provide customized time release of growth factors that simulate the natural healing process.

In some embodiments, a statin may be used as the growth factor. Statins include, but is not limited to, atorvastatin, simvastatin, pravastatin, cerivastatin, mevastatin (see U.S. Pat. No. 3,883,140, the entire disclosure is herein incorporated by reference), velostatin (also called synvinolin; see U.S. Pat. Nos. 4,448,784 and 4,450,171 these entire disclosures are herein incorporated by reference), fluvastatin, lovastatin, rosuvastatin and fluindostatin (Sandoz XU-62-320), dalvastain (EP Appln. Publn. No. 738510 A2, the entire disclosure is herein incorporated by reference), eptastatin, pitavastatin, or pharmaceutically acceptable salts thereof or a combination thereof. In various embodiments, the statin may comprise mixtures of (+)R and (−)-S enantiomers of the statin. In various embodiments, the statin may comprise a 1:1 racemic mixture of the statin.

The growth factor may contain inactive materials such as buffering agents and pH adjusting agents such as potassium bicarbonate, potassium carbonate, potassium hydroxide, sodium acetate, sodium borate, sodium bicarbonate, sodium carbonate, sodium hydroxide or sodium phosphate; degradation/release modifiers; drug release adjusting agents; emulsifiers; preservatives such as benzalkonium chloride, chlorobutanol, phenylmercuric acetate and phenylmercuric nitrate, sodium bisulfate, sodium bisulfite, sodium thiosulfate, thimerosal, methylparaben, polyvinyl alcohol and phenylethyl alcohol; solubility adjusting agents; stabilizers; and/or cohesion modifiers. In some embodiments, the growth factor may comprise sterile and/or preservative free material.

These above inactive ingredients may have multi-functional purposes including the carrying, stabilizing and controlling the release of the growth factor and/or other therapeutic agent(s). The sustained release process, for example, may be by a solution-diffusion mechanism or it may be governed by an erosion-sustained process.

The amount of growth factor, e.g., bone morphogenic protein may be sufficient to cause bone and/or cartilage growth. In some embodiments, the growth factor is rhBMP-2 and is contained in one or more carriers in an amount of from 0.05 to 2 mg per cubic centimeter of the biodegradable carrier. In some embodiments, the amount of rhBMP-2 morphogenic protein is from 2.0 to 2.5 mg per cubic centimeter (cc) of the biodegradable carrier.

In some embodiments, the growth factor is supplied in an aqueous buffered solution. Exemplary aqueous buffered solutions include, but are not limited to, TE, HEPES (2-[4-(2-hydroxyethyl)-1-piperazinyl]ethanesulfonic acid), MES (2-morpholinoethanesulfonic acid), sodium acetate buffer, sodium citrate buffer, sodium phosphate buffer, a Tris buffer (e.g., Tris-HCL), phosphate buffered saline (PBS), sodium phosphate, potassium phosphate, sodium chloride, potassium chloride, glycerol, calcium chloride or a combination thereof. In various embodiments, the buffer concentration can be from about 1 mM to 100 mM.

In some embodiments, the BMP-2 is provided in a vehicle (including a buffer) containing sucrose, glycine, L-glutamic acid, sodium chloride, and/or polysorbate 80.

In some embodiments, upon implantation of the implant, some compression of the tissue scaffold occurs that causes the buffer from the bone growth factor to leak from the carrier, which causes a higher concentrations of the growth factor (e.g., 2 mg to 6 mg of rhBMP-2 per cc of carrier) to remain on the carrier. This high concentration growth factor may lead to local transient bone resorption and excess osteoclast formation and bone breakdown. This may result in poor integration of the plug graft with surrounding host tissue and a failed repair.

In some embodiments, some compression may cause the growth factor to be absorbed and come in contact with the upper part of the tissue scaffold causing a low concentration of growth factor (e.g., 0.05 mg to 1 mg of rhBMP-2 per cc of carrier) to be exposed to the microenvironment of the defect, which may promote cartilage formation at the site the tissue scaffold is implanted. In some instances, this may be desirable especially where more cartilage tissue is needed.

### Additional Therapeutic Agents

The growth factors of the present application may be disposed on or in the tissue scaffold and/or carrier with other therapeutic agents. For example, the growth factor may be disposed on or in the carrier by electrospraying, ionization spraying or impregnating, vibratory dispersion (including sonication), nozzle spraying, compressed-air-assisted spraying, brushing and/or pouring.

Exemplary therapeutic agents include but are not limited to IL-1 inhibitors, such Kineret® (anakinra), which is a recombinant, non-glycosylated form of the human inerleukin-1 receptor antagonist (IL-1Ra), or AMG 108, which is a monoclonal antibody that blocks the action of IL-1. Therapeutic agents also include excitatory amino acids such as glutamate and aspartate, antagonists or inhibitors of glutamate binding to NMDA receptors, AMPA receptors, and/or kainate receptors. Interleukin-1 receptor antagonists, thalidomide (a TNF-α release inhibitor), thalidomide analogues (which reduce TNF-α production by macrophages), quinapril (an inhibitor of angiotensin II, which upregulates TNF-α), interferons such as IL-11 (which modulate TNF-α receptor expression), and aurin-tricarboxylic acid (which inhibits TNF-α), may also be useful as therapeutic agents for reducing inflammation. It is further contemplated that where desirable a pegylated form of the above may be used. Examples of still other therapeutic agents include NF kappa B inhibitors such as antioxidants, such as dilhiocarbamate, and other compounds, such as, for example, sulfasalazine.

Examples of therapeutic agents suitable for use also include, but are not limited to an anti-inflammatory agent, analgesic agent, or osteoinductive growth factor or a combination thereof. Anti-inflammatory agents include, but are not limited to, apazone, celecoxib, diclofenac, diflunisal, enolic acids (piroxicam, meloxicam), etodolac, fenamates (mefenamic acid, meclofenamic acid), gold, ibuprofen, indomethacin, ketoprofen, ketorolac, nabumetone, naproxen, nimesulide, salicylates, sulfasalazine [2-hydroxy-5-[-4-[C2-pyridinylamino)sulfonyl]azo]benzoic acid, sulindac, tepoxalin, and tolmetin; as well as antioxidants, such as dithiocarbamate, steroids, such as cortisol, cortisone, hydrocortisone, fludrocortisone, prednisone, prednisolone, methylprednisolone, triamcinolone, betamethasone, dexamethasone, beclomethasone, fluticasone or a combination thereof.

Suitable analgesic agents include, but are not limited to, acetaminophen, bupivicaine, fluocinolone, lidocaine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, mepiridine, methadone, morphine, nalbuphine, opium, oxycodone, papaveretum, pentazocine, pethidine, phenoperidine, piritramide, dextropropoxyphene, remifentanil, tilidine, tramadol, codeine, dihydrocodeine, meptazinol, dezocine, eptazocine, flupirtine, amitriptyline, carbamazepine, gabapentin, pregabalin, or a combination thereof.

FIG. 1A is a top view and a side sectional view of an osteochondral repair implant as it is being implanted into an osteochondral hole. A carrier having a growth factor disposed on or in it is implanted in the osteochondral hole and a tissue scaffold shown as a plug is inserted on the carrier. Upon implantation, unfortunately the plug is pressed-fit into the osteochondral hole and causes the compression of the carrier. The growth factor or buffer for the growth factor is squeezed out or leaks out of the carrier. The dose remaining on or in the biodegradable carrier may be unreliable. For example, in some embodiments, some compression of the tissue scaffold occurs that causes the buffer from the bone growth factor to leak from the carrier, which causes a higher dose of the growth factor (e.g., 2 mg or higher of rhBMP-2) to remain on the carrier. This high dose growth factor may lead to transient bone resorption and may cause failure of the implant to integrate with the surrounding tissue. In some embodiments, some compression may cause the growth factor to contact the tissue scaffold causing a low dose of growth factor (e.g., 1 mg or lower rhBMP-2) to promote cartilage formation at the site the tissue scaffold is implanted. The embodiments illustrated in FIGS. 2A-5C prevent or reduce the chance of this happening.

It will be understood by those of ordinary skill in the art that the plug can be inserted into the osteochondral hole by hand or machine (e.g., arthroscopic instrument).

FIG. 1B is a side sectional view of an osteochondral repair implant as it is implanted into an osteochondral hole. The tissue scaffold (e.g., shown as a plug) compresses against the biodegradable carrier upon implantation causing the growth factor to be squeezed or leak out. In this view the plug undergoes subsidence and drops causing an irregular outer cartilage surface, which may cause the plug not to integrate into surrounding tissue.

FIG. 2A is a top view and a side sectional view of an osteochondral hole that is drilled around the osteochondral defect. The osteochondral hole has geometric sizes in an upper portion that fits snuggly at least a portion of the tissue scaffold (e.g., shown as a plug) within the upper portion of the osteochondral hole. The lower portion has an area that fits the biodegradable carrier. Thus, the osteochondral hole is of two different sizes. One size (lower portion) has a smaller area and is configured to receive the carrier and not any significant volume of the plug. The upper portion of the hole is configured to receive the plug but the plug cannot move toward the lower region of the hole where the carrier containing the growth factor is disposed. In some embodiments, the lower hole diameter (shown as a drill recess for carrier in FIG. 2A) can be about 0.5 to about 2 mm smaller than the upper hole diameter. In some embodiments, the upper hole diameter is between about 6 to about 12 mm. By drilling the hole in this configuration, this can prevent the plug from “bottoming out” on insertion. In this way, the surgeon cannot over compress the tissue scaffold on the biodegradable carrier and release the growth factor. In some embodiments, the depth of the lower hole can be 1 mm to 3 cm and the upper hole 3-15 mm.

FIG. 2B is a side sectional view of the osteochondral repair implant that is implanted into an osteochondral hole. The osteochondral hole has geometric sizes in an upper portion that fits at least a portion of the tissue scaffold (e.g., shown as a plug) and fits it snuggly within the upper portion of the osteochondral hole. The lower portion has an area that fits the biodegradable carrier. In this way, the user upon forcing the tissue scaffold into the osteochondral hole cannot overly compress the biodegradable carrier causing unwanted growth factor leakage into the surrounding tissue.

FIG. 3 is a side sectional view of the osteochondral repair implant that is implanted into an osteochondral hole. In this view, the tissue scaffold (e.g., shown as a tapered plug) fits snuggly within the osteochondral hole. Since the tissue scaffold is tapered, the user cannot force the tissue scaffold into the osteochondral hole and thus cannot overly compress the biodegradable carrier causing unwanted growth factor leakage into the surrounding tissue.

FIG. 4A is a top view of an osteochondral hole around the osteochondral defect. FIG. 4B illustrates an embodiment of the tissue scaffold (e.g., shown as a plug) that has one mating recess and two mating projections configured to receive the biodegradable carrier or be placed around the biodegradable carrier that contains a growth factor. FIG. 4C is a side sectional view of the osteochondral repair implant that is implanted into an osteochondral hole of a cartilage surface. In this view, the tissue scaffold (e.g., shown as a plug) having one mating recess and two mating projections receives the biodegradable carrier or is placed around the biodegradable carrier. The projections and or recesses of the plug reduce the risk of the compressive force exerted on the carrier. In this way the surface area of the collagen carrier that is exposed to compressive forces upon insertion is less. Thus, it is much more difficult to compress the biodegradable carrier with the plug and cause unwanted growth factor leakage into the surrounding tissue.

It will be understood by one of ordinary skill in the art that the bottom surface of the plug may have one, two, three, four, five, six, seven, eight, etc. or more mating recesses and /or projections adaptable to be received by or placed around the carrier. Likewise, it will be understood by one of ordinary skill in the art that the top or side surface of the carrier may have one, two, three, four, five, six, seven, eight, etc. or more mating recesses and/or projections adaptable to be received by or placed around the bottom surface of the plug. The projections and or recesses of the plug and or carrier reduce the risk of the compressive force exerted on the carrier. In some embodiments, the growth factor can be disposed away from the recesses and/or projections of the plug, so that the part of the carrier that undergoes compression will be away from the plug.

FIG. 5A is a top view of an osteochondral hole around the osteochondral defect. FIG. 5B illustrates an embodiment of the tissue scaffold (e.g., shown as a plug) that has two mating recesses and one mating projection (shown as a post) configured to receive the biodegradable carrier that contains a growth factor. FIG. 5C is a side sectional view of the osteochondral repair implant that is implanted into an osteochondral hole of a cartilage surface. In this view, the tissue scaffold (e.g., shown as a plug) having two mating recesses and one mating projections (shown as a central post) receives the two projections of the biodegradable carrier. In this way, the surface area of the collagen carrier that is exposed to compressive forces upon insertion of the plug is less. Thus, it is much more difficult to compress the biodegradable carrier and cause unwanted growth factor leakage into the surrounding tissue. In some embodiments, the plugs described will provide a stable environment for the osteochondral implant and allow the carrier to release the growth factor over a period of time to produce good bone and/or cartilage tissue to repair the osteochondral defect.

### Kits

In various embodiments, a kit is provided that may include additional parts along with the tissue scaffold and/or biodegradable carrier combined together to be used to implant the osteochondral implant. The kit may include the tissue scaffold in a first compartment. The second compartment may include a biodegradable carrier and the growth factor and any other instruments needed for the implanting the osteochondral implant. A third compartment may include gloves, drapes, wound dressings and other procedural supplies for maintaining sterility during the implanting process, as well as an instruction booklet. A fourth compartment may include additional tools for implantation (e.g., drills, drill bits, bores, punches, etc.). Each tool may be separately packaged in a plastic pouch that is radiation sterilized. A fifth compartment may comprise an agent for radiographic imaging or the agent may be disposed on the tissue scaffold and/or carrier to monitor placement and tissue growth. A cover of the kit may include illustrations of the implanting procedure and a clear plastic cover may be placed over the compartments to maintain sterility.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. An osteochondral repair implant, comprising a tissue scaffold configured to allow growth of at least bone and/or cartilage and to fit within at least a portion of an osteochondral hole, the tissue scaffold comprising one or more recesses and/or projections; and a biodegradable carrier containing a growth factor, the biodegradable carrier being placed in and/or around the recesses and/or projections of the tissue scaffold to reduce compression of the biodegradable carrier by the tissue scaffold.

2. An osteochondral repair implant according to claim 1, wherein the tissue scaffold comprises at least one of collagen, a resorbable polymer, gelatin, a resorbable ceramic, hyaluronic acid, chitosan or combinations thereof.

3. An osteochondral repair implant according to claim 1, wherein the tissue scaffold comprises an osteochondral plug, pin, peg, or cylinder of autograft, allograft or xenograft bone and/or cartilage and is configured to fit snugly in the osteochondral hole.

4. An osteochondral repair implant according to claim 1, wherein (i) the tissue scaffold comprises a tapered osteochondral plug or (ii) the tissue scaffold comprises one or more mating recesses and/or projections; and the biodegradable carrier has one or more opposing mating recesses and/or projections configured to receive the one or more mating recesses and/or projections of the tissue scaffold to reduce compression of the biodegradable carrier by the tissue scaffold.

5. An osteochondral repair implant according to claim 1, wherein (i) the tissue scaffold comprises a tapered osteochondral plug configured to fit snugly in an upper portion of the osteochondral hole without substantially moving toward a lower portion of the osteochondral hole and compressing the biodegradable carrier and (ii) the biodegradable carrier is configured to fit within the lower portion of the osteochondral hole and contact a bottom surface of the osteochondral plug.

6. An osteochondral repair implant according to claim 1, wherein the tissue scaffold comprises at least one bottom surface having one or more recesses and/or projections and the biodegradable carrier being placed in and/or around the projections so as to allow the biodegradable carrier to remain substantially in an uncompressed state upon insertion of the tissue scaffold in the osteochondral hole.

7. An osteochondral repair implant according to claim 1, wherein the implant reduces leakage of the growth factor from the biodegradable carrier by reducing compression of the biodegradable carrier.

8. An osteochondral repair implant according to claim 1, wherein the biodegradable carrier comprises at least one of collagen, a resorbable polymer, gelatin, a resorbable ceramic or combinations thereof.

9. An osteochondral repair implant according to claim 1, wherein (i) the tissue scaffold comprises an osteochondral plug; (ii) the biodegradable carrier comprises collagen; and (iii) the growth factor comprises bone morphogenic protein-2.

10. An osteochondral repair implant, comprising a osteochondral plug configured to allow growth of at least bone and/or cartilage and to fit within at least a portion of an osteochondral hole, the osteochondral plug comprising one or more recesses and/or projections; and a biodegradable carrier containing a growth factor, the biodegradable carrier being placed in and/or around the recesses and/or projections of the osteochondral plug to reduce compression of the biodegradable carrier by the osteochondral plug and leakage of the growth factor from the biodegradable carrier.

11. An osteochondral repair implant according to claim 10, wherein the osteochondral plug comprises autograft, allograft or xenograft bone and/or cartilage and is configured to fit snugly in the osteochondral hole.

12. An osteochondral repair implant according to claim 10, wherein the osteochondral plug comprises a tapered lower portion to fit snugly within at least a portion of an osteochondral hole to reduce compression of the biodegradable carrier and leakage of the growth factor from the carrier.

13. An osteochondral repair implant according to claim 10, wherein (i) the osteochondral plug is configured to fit snugly in an upper portion of the osteochondral hole without substantially moving toward a lower portion of the osteochondral hole and compressing the biodegradable carrier and (ii) the biodegradable carrier is configured to fit within the lower portion of the osteochondral hole and contact a bottom surface of the osteochondral plug.

14. An osteochondral repair implant according to claim 10, wherein the osteochondral plug comprises at least one bottom surface having one or more recesses and/or projections and the biodegradable carrier being placed in and/or around the recesses and/or projections so as to allow the biodegradable carrier to remain substantially in an uncompressed state upon insertion of the osteochondral plug in the osteochondral hole.

15. An osteochondral repair implant according to claim 10, wherein the implant reduces leakage of the growth factor from the biodegradable carrier by reducing compression of the biodegradable carrier when pressure is applied to the plug.

16. An osteochondral repair implant according to claim 10, wherein the biodegradable carrier comprises at least one of collagen, a resorbable polymer, gelatin, a resorbable ceramic or combinations thereof.

17. An osteochondral repair implant according to claim 10, wherein (i) the osteochondral plug is bioresorbable or (ii) the osteochondral plug comprises one or more mating recesses and/or projections; and the biodegradable carrier has one or more opposing mating recesses and/or projections configured to receive the one or more mating recesses and/or projections of the osteochondral plug to reduce compression of the biodegradable carrier by the osteochondral plug.

18. An osteochondral repair implant according to claim 10, wherein (i) the osteochondral plug comprises autograft, allograft or xenograft bone and/or cartilage; (ii) the biodegradable carrier comprises collagen; and (iii) the growth factor comprises bone morphogenic protein-2.

19. A method for repairing an osteochondral defect in a patient in need of such treatment, the method comprising: forming a hole in the bone and/or cartilage in an osteochondral area in need of repair, the hole having an upper portion configured to receive a tissue scaffold and a lower portion configured to receive a biodegradable carrier containing a growth factor; inserting into the hole the biodegradable carrier so as to fill part or all of the lower portion of the hole with the biodegradable carrier and inserting into the hole a tissue scaffold so as to fill part or all of the upper portion of the hole with the biodegradable carrier to stack the tissue scaffold on the biodegradable carrier without compressing the biodegradable carrier and causing substantial leakage of the growth factor therefrom.

20. A method for repairing an osteochondral defect according to claim 19, wherein the upper portion of the hole is wider than the lower portion of the hole so that the tissue scaffold cannot substantially move toward the lower portion of the hole upon pressure to compress the biodegradable carrier.
